(19) United States
(12) Patent Application Publication
(10) Pub. No.: US 2016/0150982 A1
(43) Pub. Date: Jun. 2, 2016

(54) WEARABLE RESPIRATORY INDUCTANCE PLETHYSMOGRAPHY DEVICE AND METHOD FOR RESPIRATORY ACTIVITY ANALYSIS

(71) Applicant: Carre Technologies Inc., Montreal (CA)

(72) Inventors: Jean-François ROY, Montreal (CA); Pierre-Alexandre FOURNIER, Montreal (CA); Charles ROBILLARD, Saint-Lazare (CA); Robert CORRIVEAU, Saint-Bruno (CA); Simon DUBEAU, Montreal (CA); Antoine GAGNE-TURCOTTE, Montreal (CA); David KHOUYA, St-Eustache (CA)

(21) Appl. No.: 14/955,749

(22) Filed: Dec. 1, 2015

(30) Foreign Application Priority Data
Dec. 2, 2014 (CA) 2,872,754
Jul. 9, 2015 (CA) 2,896,498

(51) Int. Cl. A61B 5/0205; A61B 5/11; A61B 5/00

(52) U.S. Cl. CPC A61B 5/02055; A61B 5/6804; A61B 5/7278; A61B 5/11; A61B 5/4818; A61B 5/021

(57) ABSTRACT

It is described a system and a method for respiratory activity analysis comprising the use of Respiratory Inductance Plethysmography (RIP). In particular, a wearable system for extracting physiological parameters of a person by measuring at least one plethysmographic signal is disclosed. The system comprises: a wearable garment fitting a body part of the person; at least one wire supported by or embedded into the garment, each wire forming a loop around the body part when the person wears the garment for measuring a plethysmographic signal; and an electronic device supported by or fixed on the garment and including a Colpitts oscillator connected to each wire loop, wherein the Colpitts oscillator has an optimal frequency band from 1 MHz to 15 MHz for extracting the plethysmographic signal measured by each wire, the electronic device converting analog information measured by the Colpitts oscillator into digital analyzable information.

### CROSS-REFERENCE TO RELATED APPLICATIONS

The present patent application claims the benefits of priority of commonly assigned Canadian Patent Application no. 2,872,754, entitled “LOW-POWER RESPIRATORY INDUCTANCE PLETHYSMOGRAPHY DEVICE, INTELLIGENT GARMENTS OR WEARABLE ITEMS EQUIPPED THEREWITH AND A METHOD FOR RESPIRATORY ACTIVITY ANALYSIS” and filed at the Canadian Intellectual Property Office on Dec. 2, 2014; and Canadian Patent Application no. 2,896,498, entitled “WEARABLE RESPIRATORY INDUCTANCE PLETHYSMOGRAPHY DEVICE AND METHOD FOR RESPIRATORY ACTIVITY ANALYSIS”, filed at the Canadian Intellectual Property Office on Jul. 9, 2015 and opened to public inspection in advance on Oct. 21, 2015. The content of these applications is incorporated herewith by reference.

### FIELD OF THE INVENTION

The present invention relates to the field of ambulatory and non-invasive monitoring of an individual's physiological parameters. In particular, it is described a system and a method for respiratory activity analysis comprising the use of Respiratory Inductance Plethysmography (RIP) sensor using an optimal Colpitts oscillator configuration for an efficient human body measurement. The system can be a garment or other wearable item.

### BACKGROUND

Physiological sensors have long been known and widely used for medical and health related applications. Various physiological sensors embedded in textile or garments, sometimes called portable or wearable sensors, have been described before in publications and patents (Portable Blood Pressure in U.S. Pat. No. 4,889,132; Portable device for sensing cardiac function in U.S. Pat. No. 4,928,690; Heart rate monitor in garment in U.S. Pat. No. 7,680,523 B2). The term “wearable sensors” is now commonly used to describe a variety of body-worn sensors to monitor activity, environmental data, body signals, biometrics, health related signals, and other types of data. Garment may include a stretchable harness such as in U.S. Pat. No. 8,818,478 B2.

As used herein, “plethysmography”, and its derivative words, is the measurement of changes in volume within an organ or whole body, or a cross-sectional area of the body when the body's is constant in height. “Inductive plethysmography” is a plethysmographic measurement based on determination of an inductance or a mutual inductance. A “plethysmographic signal” is a signal generated by plethysmography, and specifically by inductive plethysmography. The cross-sectional area of the body measured by a plethysmograph may include, singly or in combination, the chest, abdomen, neck, or arm.

The inductance sensor may be as simple as a conductive loop wrapped around the body cross-section. The loop is attached to a close-fitting garment that expands and contracts with the body cross-section. As the body cross-section expands and contracts, the area enclosed by the loop also expands and contracts thereby changing the inductance of the loop. The inductance change of the loop may be converted to an electrical signal using methods known to one of skill in the electrical art.

If the loop is placed around the chest, the changes in the loop inductance may be correlated to respiration volumes. For example, U.S. Pat. No. 4,308,872 issued Jan. 5, 1982 and titled “Method and Apparatus for Monitoring Respiration,” discloses a method and apparatus for monitoring respiration volumes by measuring variations in the patient's chest cross sectional area.

Respiratory Inductive Plethysmography (RIP) is based on the analysis of the movement of a cross-section of the human torso with a low-resistance conductive loop using conductive textile or knitted warn, wire within an elastic band or braid, a loose wire within a textile tunnel or any conductive material in a configuration that makes it extensible. The extensibility is needed to follow the body as it changes shape due to breathing, movement, or other activities that can modify the body shape and volume.

Many patents and articles mention methods to use RIP sensors such as “Development of a respiratory inductive plethysmography module supporting multiple sensors for wearable systems” by Zhang Z, et al., Sensors 2012; 12, 13167-13184. It is hard to obtain good percentage of effective data as stated at page 23 of the article entitled “A Wearable Respiration Monitoring System Based on Digital Respiratory Inductive Plethysmography”, Bulletin of Advanced Technology Research, Vol. 3, No. 9/September 2009, where only 83% of effectiveness is achieved.

Many types of oscillators have been proposed for RIP sensing and used with different configurations. Noise and artifacts due to movement or other causes are common when RIP sensing is used in a garment or other wearable item. The system must be designed to tolerate noise and artifacts and be able to filter many of them to provide accurate breathing measurements.

Using data from one or many RIP sensors, analysis can provide major metrics such as Respiratory Rate, Tidal Volume and Minute ventilation, Fractional inspiratory time (T inhale, T exhale), and other information about the physiological and psychological state of the person or animal wearing the garment or the wearable item.

Determining signal quality and data quality for wearable sensors is very challenging. The assessment of signal and data quality is an important part of many high-level analysis algorithms, visual presentation of the data, and interpretation of the data in general.

### SUMMARY OF THE INVENTION

The invention is first directed to a wearable system for extracting physiological parameters of a person by measuring at least one plethysmographic signal. The system comprises:
a wearable garment fitting a body portion of the person;

at least one wire supported by or embedded into the garment, each wire forming a loop around the body part when the person wears the garment for measuring a plethysmographic signal; and

an electronic device supported by or fixed on the garment and including a Colpitts oscillator connected to each wire loop;

wherein the Colpitts oscillator has an optimal frequency band from 1 MHz to 15 MHz for extracting the plethysmographic signal measured by each wire, the electronic device converting analog information measured by the Colpitts oscillator into digital analyzable information.

The invention is also directed to a method for extracting physiological parameters of a person, the method comprising the steps of:
a) providing a wearable garment, the garment fitting a body portion of the person;
b) measuring at least one plethysmographic signal using at least one wire supported by or embedded into the garment, each wire forming a loop around the body part;
c) extracting the plethysmographic signal measured by each wire using an electronic device supported by the garment, the electronic device including a Colpitts oscillator connected to each wire and having an optimal frequency band from 1 MHz to 15 MHz; and
d) converting analog information measured by the Colpitts oscillator into digital analyzable information.

The invention is further directed to the use of the wearable system as disclosed herein, for extracting physiological parameters of a person by measuring at least one plethysmographic signal. Preferably, the physiological parameters extracted by the system are breathing metrics selected from the group consisting of respiratory rate, tidal volume, minute ventilation and fractional inspiratory time.

The invention is further directed to the use of the wearable system as disclosed herein, for detecting and characterizing physical conditions selected from the group consisting of talking, laughing, crying, hiccups, coughing, asthma, apnea, sleep apnea, stress related apnea, relaxation exercise, breathing cycle symmetry, and pulmonary diseases.

The invention is yet further directed to the use of the wearable system as disclosed herein, for detecting and characterizing heart activities selected from the group consisting of heart rate, body movements and activities. Preferably, the body activities are walking and running.

When the user put the garment on, such as a shirt or T-shirt, the wire loops (also named RIP sensors) are then placed around the user body. The garment minimizes the variation in the positioning of the RIP sensor(s) for a better accuracy and repeatability.

Once the low-powered electronic device in connected to the shirt, the Colpitts oscillator circuit is activated to begin the measurement, it measures the area surrounded by the RIP sensor, like a slice of the body. When the user breathes, the sensor move and the area to measure change, by doing so the oscillator circuit change slightly his oscillation frequency reflecting the impedance changes.

Garments, such as shirts, from a complete size set will all have a different inductance with the same oscillator circuit. The electronic device measures main frequency and the delta frequency from the oscillator to estimate the breathing rate, amplitude and volume.

Advantageously, the garment is easy to put allowing to precisely place the sensors providing reliability and accuracy of the Colpitts even for small movement. The garment does not hinder the movements of the person wearing it while providing excellent quality measurements of biometric signals.

### BRIEF DESCRIPTION OF THE DRAWINGS

The description makes reference to the accompanying drawings wherein like reference numerals refer to like parts throughout the several views and wherein:

FIG. 1 is a diagram the amplitude versus the frequency and the current for the same frequency for a Colpitts oscillator showing the defined optimal frequency range of the Colpitts oscillator when measured around a human body.

FIG. 2 is a high level diagram showing how a battery power Colpitts oscillator can be connected to a garment to do signal acquisition. FIG. 2 also shows the digital signal processing (DSP) that could be performed to provide useful data statistics and filtered signals.

FIG. 3 as an example of the state machine for algorithm based on two RIP sensors data to extract the breathing rate, the minute ventilation and the tidal volume.

FIG. 4 is an example of how the wearable garment artifacts can be filtered out.

FIG. 5 show a Smith chart result of the RIP sensor stimulated between 1 MHz and 10 MHz showing the good linearity response of the Colpitts oscillator.

FIG. 6 shows garments that use the present system to connect textiles sensors for heart and breathing monitoring to an electronic device with an accelerometer and a Bluetooth wireless connection. The electronic device also contains analog and digital filters and amplifiers, a microprocessor device, solid-state memory storage, sensor circuits, power management circuits, buttons, and other circuits.

FIG. 7 shows an example of a garment that includes RIP sensors, electrical, thermal, and optical sensors for cardiac monitoring, breathing monitoring, blood pressure monitoring, skin temperature and core temperature monitoring to an electronic device with position and movement sensors and a wireless data connection.

### DETAILED DESCRIPTION OF PREFERRED EMBODIMENTS

The foregoing and other features of the present invention will become more apparent upon reading of the following non-restrictive description of examples of implementation thereof, given by way of illustration only with reference to the accompanying drawings.

Low power sensing is a domain with many technological challenges for designers and manufacturers of e-textile solutions, intelligent garments, wearable sensors, and multi-parameter wearable connected personal monitoring systems.

As aforesaid, the present invention first concerns a wearable system for extracting physiological parameters of a person by measuring at least one plethysmographic signal. The system first comprises a wearable garment fitting a body part of the person.

By “garment”, it is understood any sort of garment or clothing that can be worn by a person. The garment when worn should fit sufficiently the body of the person to be in close contact with the body to follow the movement of the body. Adjusted T-shirt is particularly adapted but any other sort of clothing can be used as long as it fits the body. A belt strapped or a tube around the torso can also be used instead of a T-shirt. The garment can be made of any kinds of fabrics. Preferably, the wearable system according to the invention is washable.

The system also comprises at least one wire supported by or embedded into the garment. Each wire forms a loop around the body part, when the person wears the garment for measuring a plethysmographic signal.

By “supported”, it is to be understood that the RIP wire is the RIP wire loop could be woven, knitted, laminated, glued, stitched or even soldered to the garment. By “embedded”, it is to be understood that the wire loop is enclosed in a protective element supported by the garment. It can be a overstitching into the fabric or a guiding portion as detailed below.

As aforesaid, by “plethysmography”, and its derivative words, is meant the measurement of a cross-sectional area of a body when the body is constant or almost constant in height. By “Inductive plethysmography”, it is meant a plethysmographic measurement based on determination of an inductance or a mutual inductance. By “plethysmographic signal”, it is meant a signal generated by plethysmography, and specifically by inductive plethysmography. The cross-sectional area of the body measured by a plethysmograph may include, singly or in combination, the chest, abdomen, neck, or arm.

The system also comprises a low-powered electronic device supported by or fixed on the garment. The device can be attached to the garment, embedded into the garment such in an open or close pocket thereof. The device includes a Colpitts oscillator connected to each wire loop. The Colpitts oscillator was invented in 1918 by Edwin Colpitts, and reference can be made to U.S. Pat. No. 1,624,537.

A Colpitts oscillator is one of a number of designs for LC oscillators, electronic oscillators that use a combination of inductors (L) and capacitors (C) to produce an oscillation at a certain frequency. The distinguishing feature of the Colpitts oscillator is that the feedback for the active device is taken from a voltage divider made of two capacitors in series across the inductor. A change in the cross section of the body measured by the RIP sensor causes the

Colpitts oscillator to change its oscillating frequency. A digital and/or analog electronic circuit is used to measure the frequency, the change in frequency, and/or the rate of change of the frequency of the Colpitts oscillator.

The Colpitts oscillator of the system according to the present invention has an optimal frequency band from 1 MHz to 15 MHz in order to extract the plethysmographic signal measured by each wire. The electronic device then converts analog information measured by the Colpitts oscillator into digital analyzable information.

According to a preferred embodiment, the system may further comprise at least one connector embedded into the garment for connecting the Colpitts oscillator to each wire loop. Any sorts of connector know in the art for this application can be used, such as the one developed and patented by the Application with Canadian patent No. CA 2,867,205, the content of which is incorporated herein by reference.

According to a preferred embodiment, the garment may comprise at least one guiding portion embedded into the garment. Since each guiding portion is adapted for receiving and maintaining the wires in a predetermined position around the body portion, the number of guiding portion depends on the numbers of wire loops present in the system. The guiding portion can be of any kind known in the art, such as an overstitching in the fabric of the garment.

According to a preferred embodiment, when the body portion is the torso of the person wearing the garment, the system may then comprise a first loop of wire placed around a thoracic section of the torso and a second loop of said wires being placed around an abdominal section of the person; allowing as such to measure the breathing frequency and/or frequency change of the person. Each wire loop is preferably constructed using a conductive material in a configuration that makes the garment extensible textile that fits the wearer body.

According to a preferred embodiment, the system may further comprise a power source or generator for powering the Colpitts oscillator and electronic device. The power source may be external and adapted to be worn by the user, such as in a pocket, or embedded into the garment. More preferably, the power source is embedded in a section of the garment, such as a pocket or an overstitching. The power source can be a battery or any sort of power source adapted to power the electronic device and Colpitts oscillator. Energy harvesting or scavenging systems known in the art can be also used to provide power, such as those using Peltier effect.

According to a preferred embodiment, the Colpitts oscillator is adapted to be turned on and off a plurality of times per second according to a frequency sampling to extend the power life of the power source.

According to a preferred embodiment, the low-powered electronic device is a digital processing device for converting analog information into digital information by applying at least one algorithm to analyze the information. Preferably, the low-powered electronic device may be in communication with a smart phone or a computer using a wireless connection, such as but not limited to a Bluetooth connection.

According to a preferred embodiment, the system may further comprise at least one sensor supported or embedded into the garment. Any sensors known in the art for measuring body temperature, blood pressure and/or heart beat frequency can be used.

According to a preferred embodiment, the physiological parameters extracted by the system may be breathing metrics such as, but not limited to, respiratory rate, tidal volume, minute ventilation and fractional inspiratory time.

According to a preferred embodiment, the system may also provide metrics to detect and characterize physical conditions such as, but not limited to, talking, laughing, crying, hiccups, coughing, asthma, apnea, sleep apnea, stress related apnea, relaxation exercise, breathing cycle symmetry, and pulmonary diseases. The system may also provide metrics to detect and characterize heart activities such as, but not limited to, heart rate, body movements and activities, such as, but not limited to walking and running.

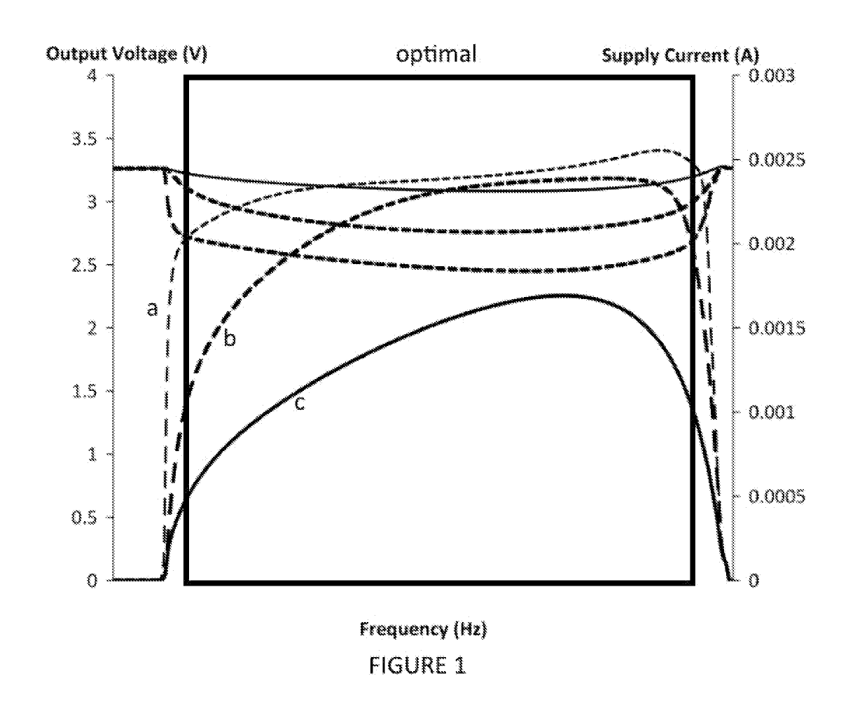

FIG. 1 is a diagram showing the amplitude versus the frequency and the current for the same frequency range for a Colpitts oscillator. An optimal frequency range has been determined and implemented for the impedance loop. This range covers but is not restricted to the frequency band from 1 MHz to 15 MHz. This frequency range has been found to be optimal for the human body composition. The frequency is optimal for maximum precision for a garment or object equipped therewith. The figure shows 3 simulations results with different RIP loop inductance values in the valid range for torso measurements: curves dot-line (a=1.8 μH or microhenry), double-line (b=2 μH) and the plain=-line (c=232 μH).

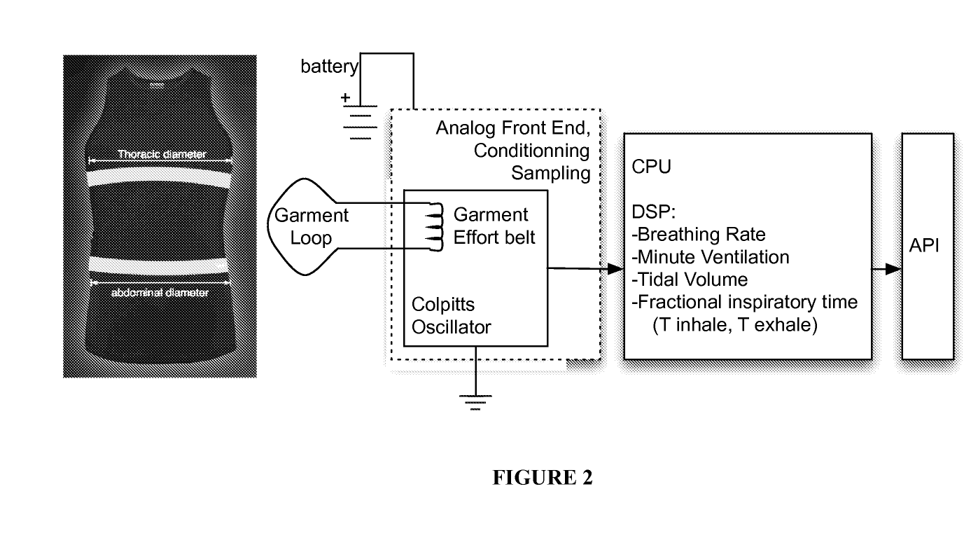

Preferably, the low resistivity impedance effort system of the invention comprises the use of a wire loop placed within the wearable garment. The impedance loop used is preferably a wire strategically placed in a textile guide incorporated into the garment or object fabric (as exemplary shown in FIG. 2). The loop goes from one connector contact to another going around the torso of the wearer. The wearable device computes the statistics such as breathing rate or breathing volume or tidal volume or the fractional inspiration time.

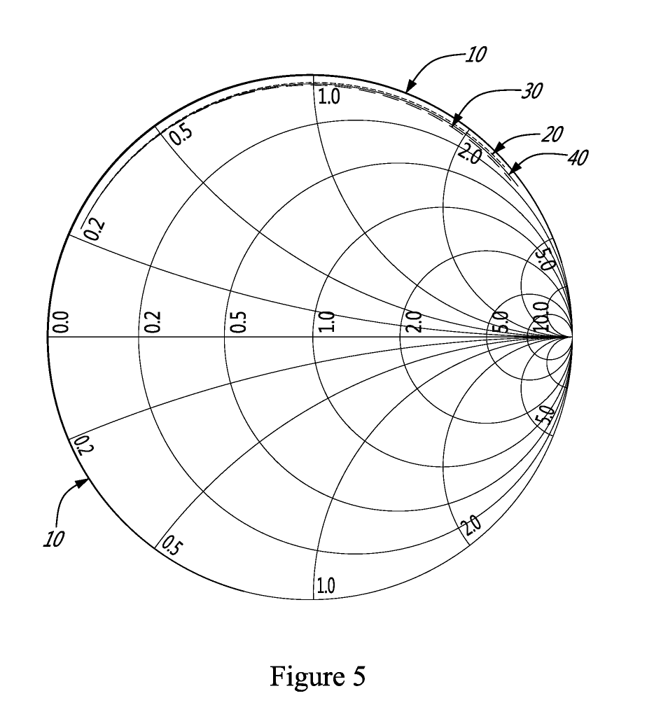

FIG. 5 shows a Smith chart result of the RIP sensor stimulated between 1 MHz and 15 MHz, of impedance of a garment using a Vectorial analyzer HP 8753 300 kHz-3.0 GHz [Canal 1 Ind. Att1=0 dB; Att2=0 dB; R/Z0 series:G/Y0 paral. Scale factor=1.00 U FS; IF=3.00 kHz; Z0=50.0]

The results of FIG. 5 are presented in the Table below:

Foper = 4.015 MHz

| Reference | | | | | |
|---|---|---|---|---|---|
| on FIG. 5 | Samples | L₀ | Z₀ | Xl | R |
| 10 | Body form with air | 1.88 μH | 47.38 | 47.35 | 1.61 |
| | (maximum diameter) | | | | |
| 20 | Body form with air | 1.89 μH | 47.65 | 47.72 | 1.64 |
| | (maximum diameter) | | | | |
| 30 | Human body | 1.95 μH | 49.24 | 49.21 | 1.74 |
| 40 | Same garment as 10 | 2.05 μH | 51.69 | 51.66 | 1.87 |
| | and 20 but with a | | | | |
| | human body | | | | |

[Minimal frequency = 1.00 MHz; Maximum frequency = 10.00 MHz; Electric delay= 0.000 s; dφ = 0.00°; Sweeping = 100.00 ms; Type: VS Freq. Lin Mode: S11 − Conversion = none]

The inductance variation due to movement of the electronic device, such as the RIP, is very small but more efficient. Movement of the body part produces Delta Inductance, then producing a delta frequency, then producing a delta amplitude, then producing n bit sampling.

The Colpitts oscillator in the frequency range from 1 MHz to 15 MHz is proven to be linear. FIG. 5 shows an excellent linearity with a resulting impedance around 2 micro Henry (μH).

To reduce power consumption further, the Colpitts oscillator can be turned ON and OFF many times per second. Sufficient ON time is needed to be able to sample the frequency of the Colpitts oscillator.

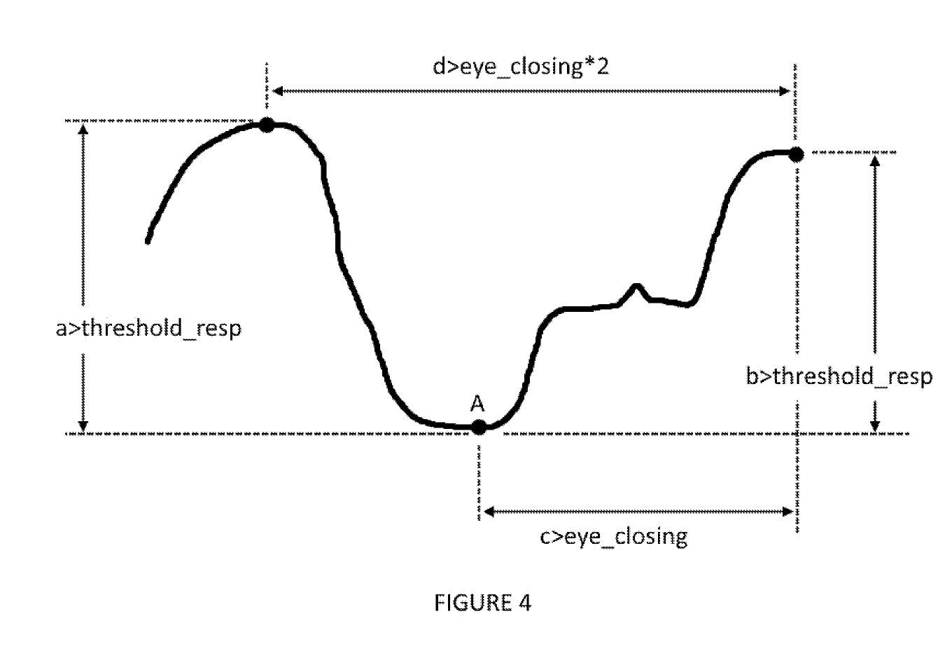

As described in FIG. 4, two criteria are considered to detect inspiration/expiration. One is the adaptive filter threshold; the other is the eye closing (the inhibition period). In FIG. 4, an expiration is found when the condition (point A, minimum). It also applies to detection of inspiration but searching for maximum.

One example of adaptive Threshold resp is shown in FIG. 4, where:

25% of the average duration of the 4 last expirations

5≧Threshold_resp≧50

One example of adaptive Eye_closing is also shown in FIG. 4, where:

25% of the average duration of the 4 last respiration (i.e. inspiration+expiration)

16≧Eye_closing≧256 (at 128 Hz, thus 0.125-2 s)

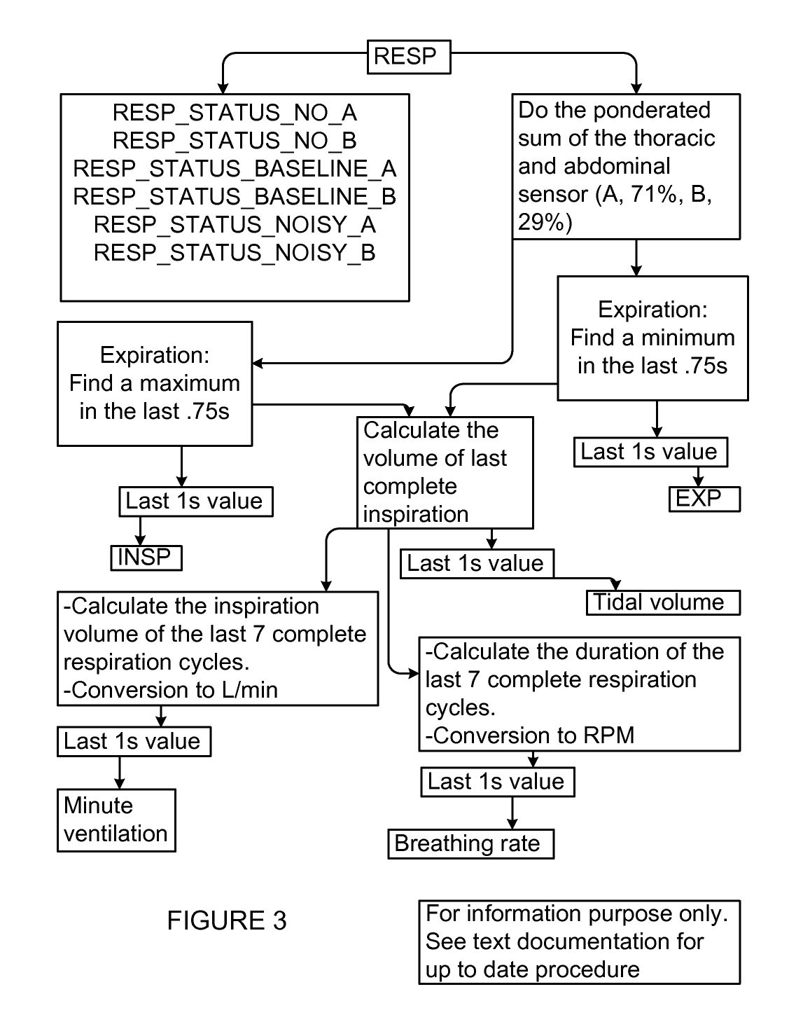

The algorithm described is FIG. 3 shows an example of adaptive filtering with two RIP bands, using a weighted sum of the thoracic and abdominal signal for inspiration/expiration detection usage to extract minute ventilation, breathing rate, tidal volume and fractional inspiratory time (INSP: T inhale, EXP: T exhale). RESP is the sensing input coming from the Colpitts oscillator. Signal quality assessment is performed to validate input regarding the noise status of the sensor, its baseline linearity check and general status such connector connect/disconnect detection.

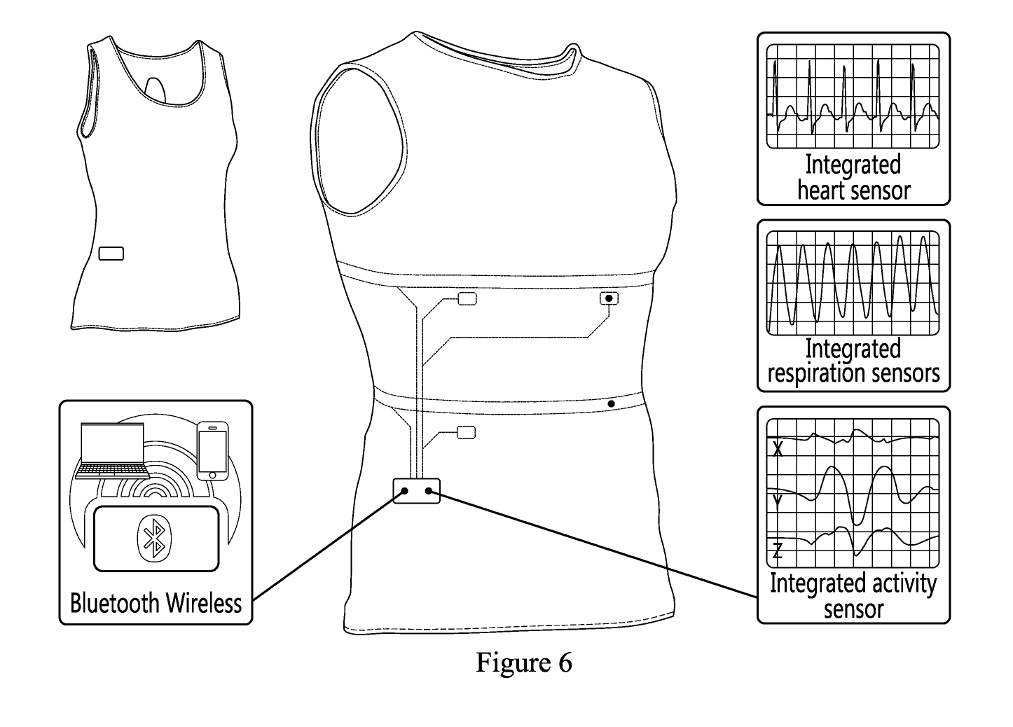

FIG. 6 shows an example of the RIP sensor integration in the wearable system. The sensors are normally passive and become active only once they are connected to the active electronic analog front end. Two RIP sensors are placed on a shirt, one on the torso one on the abdomen. Three textile electrodes are also placed, one differential input (ECG lead I) and one reference. All sensors electrical signal lines are interconnected through the connector to the small wireless apparatus. An apparatus comprising a 3-axis accelerometer motion sensor, local memory for data, processing capabilities to analyze data in real-time, and Bluetooth communication capabilities, is used to communicate with smart phones and computers. The data is processed and analyzed in the device in order to transmit only what is important to minimize power consumption. The smart phone and computer network connectivity make possible remote server communication, which can provide automatic physiological data analysis services and help with the interpretation of physiological signals.

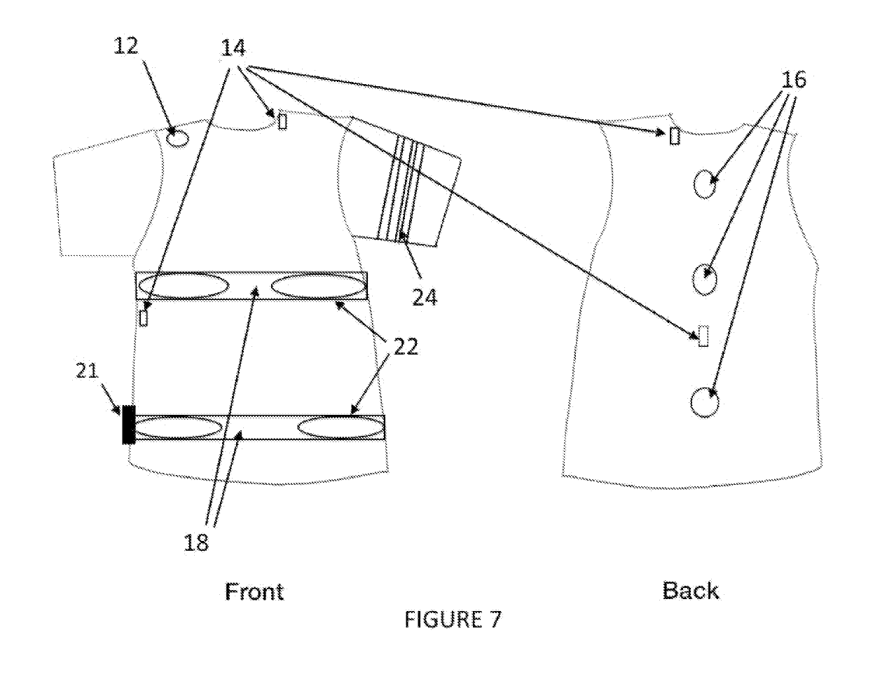

FIG. 7 is another wearable garment example where many more sensors are integrated into the fabric. For each sensor a different wiring technique can be used such as wires, knitted conductive fibers, laminated conductive textile, optic fiber and/or polymer. Sensors can be strategically placed to perform good quality biometric measurements. FIG. 7 shows a garment in accordance with one embodiment of the invention having two RIP band sensors (18), four textile electrodes ECG (22), a caught pressure sensor on the left arm (24), four temperature sensors (14), three position and orientation sensors (16), and an optical spectroscopy sensor (12).

Other type of sensors such as galvanic skin response (GSR), stretch sensors for structural sensing and others can be used. The garment also comprises an electronic device (21), preferably a low-powered electronic device, for converting analog information measured by the Colpitts oscillator into digital analyzable information.

### EXAMPLE 1

### Shirt for Men—Small size

Duty cycle of 50%, with a time ON for the breathing circuit of 20 ms.

Oscillation frequency: 4.3 MHz

### EXAMPLE 2

### Shirt for Men—Large size

Duty cycle of 50%, with a time ON for the breathing circuit of 20 ms.

Oscillation frequency: 5.4 MHz

The oscillation frequency varies between the two examples above due to the shirt's impedance with the wire length of different size.

The present invention also concerns a method for extracting physiological parameters of a person. The method comprises at least the followings steps:
a) providing a wearable garment, the garment fitting a body part of the person;
b) measuring at least one plethysmographic signal using at least one wire supported by or embedded into the garment, each wire forming a loop around the body part;
c) extracting the plethysmographic signal measured by each wire using a low-powered electronic device supported by the garment, the electronic device including a Colpitts oscillator connected to each wire and having an optimal frequency band from 1 MHz to 15 MHz; and
d) converting analog information measured by the Colpitts oscillator into digital analyzable information.

According to a preferred embodiment, the method may further comprise the step of connecting the Colpitts oscillator to each wire using at least one connector embedded into the garment. As aforesaid, the number of connector will depend on the number of wire loop to be connected to the electronic device.

According to a preferred embodiment, the method may further comprise the step of maintaining each wire in a predetermined position around the body portion using a guiding portion embedded into the garment.

According to a preferred embodiment, when the body portion is the torso of the person wearing the garment, the method may then comprise the steps of:

providing a first loop of said wires around a thoracic section of the torso;

providing a second loop of said wires around an abdominal section of the person; and

measuring a breathing frequency and/or frequency change of the person.

According to a preferred embodiment, the method may further comprise the step of making each wire extensible by using an extensible configuration of a conductive material.

According to a preferred embodiment, the method may further comprise the step of powering the Colpitts oscillator and low-powered electronic device using a power source. Preferably, the electricity power source is embedded into the garment. Preferably, the electricity power source may be a battery.

According to a preferred embodiment, the method may further comprise the step of turning on and off the Colpitts oscillator a plurality of times per second according to a frequency sampling to extend a power life of the power source.

Preferably, in the method according to the present invention, the step of converting analog information into digital information further comprises the step of analyzing the information by applying at least one algorithm.

According to a preferred embodiment, the method may further comprise the step of communicating the information from the electronic device to a smart phone or a computer using a wireless connection. The wireless connection may be a Bluetooth connection, but other known wireless communications can be used.

According to a preferred embodiment, the method may further comprise the step of measuring body temperature, blood pressure and/or heart beat frequency using at least one sensor embedded into the garment and connected to the electronic device.

Preferably, the physiological parameters extracted by the application of the present method are breathing metrics such as, but not limited to respiratory rate, tidal volume, minute ventilation and fractional inspiratory time.

According to a preferred embodiment, the method may further comprise the step of detecting and characterizing physical conditions such as, but not limited to talking, laughing, crying, hiccups, coughing, asthma, apnea, sleep apnea, stress related apnea, relaxation exercise, breathing cycle symmetry, and pulmonary diseases.

According to a preferred embodiment, the method may further comprise the steps of detecting and characterizing heart activities such as but not limited to heart rate, body movements and activities, such as walking and running.

The scope of the claims should not be limited by the preferred embodiments set forth in the examples, but should be given the broadest interpretation consistent with the description as a whole.

What is claimed is:

1. A wearable system for extracting physiological parameters of a person by measuring at least one plethysmographic signal, the system comprising:
a wearable garment fitting a body portion of the person;
at least one wire supported by or embedded into the garment, each wire forming a loop around the body part when the person wears the garment for measuring a plethysmographic signal; and
an electronic device supported by or fixed on the garment and including a Colpitts oscillator connected to each wire loop;
wherein the Colpitts oscillator has an optimal frequency band from 1 MHz to 15 MHz for extracting the plethysmographic signal measured by each wire, the electronic device converting analog information measured by the Colpitts oscillator into digital analyzable information.

2. The system of claim 1, further comprising at least one connector embedded into the wearable garment for connecting the Colpitts oscillator to each wire loop.

3. The system of claim 1, wherein the wearable garment comprises at least one guiding portion embedded into the garment, each guiding portion being adapted for receiving and maintaining one of said at least one wire in a predetermined position around the body portion.

4. The system of claim 1, wherein the body portion is the torso of the person wearing the wearable garment, the system then comprising a first loop of said wires being placed around a thoracic section of the torso and a second loop of said wires being placed around an abdominal section of the person; for measuring a breathing frequency and/or frequency change of the person.

5. The system of claim 1, wherein each wire loop is constructed using a conductive material in a configuration that makes the wearable garment extensible.

6. The system of claim 1, further comprising a power source for powering the Colpitts oscillator and the electronic device.

7. The system of claim 6, wherein the power source is embedded into the garment.

8. The system of claim 6, wherein the power source is a battery or an energy harvesting system.

9. The system of claim 6, wherein the Colpitts oscillator is adapted to be turned on and off a plurality of times per second according to a frequency sampling to extend a power life of the power source.

10. The system of claim 1, wherein the electronic device is a digital processing device for converting analog information into digital information by applying at least one algorithm to analyze the information.

11. The system of claim 1, wherein the electronic device is in communication with a smart phone or a computer using a wireless connection.

12. The system of claim 11, wherein the wireless connection is a Bluetooth connection.

13. The system of claim 1, further comprising at least one sensor for measuring body temperature, blood pressure and/or heart beat frequency.

14. The system of claim 1, wherein the physiological parameters extracted by the system are breathing metrics selected from the group consisting of respiratory rate, tidal volume, minute ventilation and fractional inspiratory time.

15. The system of claims 1, wherein the system also provide metrics to detect and characterize physical conditions selected from the group consisting of talking, laughing, crying, hiccups, coughing, asthma, apnea, sleep apnea, stress related apnea, relaxation exercise, breathing cycle symmetry, and pulmonary diseases.

16. The system of claims 1, wherein the system also provides metrics to detect and characterize heart activities selected from the group consisting of heart rate, body movements and activities.

17. The system of claim 16, wherein the body activities are walking and running.

18. The system of claim 1, wherein the frequency of the Colpitts oscillator is about 4.3 MHz.

19. The system of claim 1, wherein the frequency of the Colpitts oscillator is about 5.4 MHz.

20. A method for extracting physiological parameters of a person, the method comprising the steps of:
a) providing a wearable garment, the wearable garment fitting a body portion of the person;
b) measuring at least one plethysmographic signal using at least one wire supported by or embedded into the garment, each wire forming a loop around the body part;
c) extracting the plethysmographic signal measured by each wire using a low-powered electronic device supported by the garment, the electronic device including a Colpitts oscillator connected to each wire and having an optimal frequency band from 1 MHz to 15 MHz; and
d) converting analog information measured by the Colpitts oscillator into digital analyzable information.
